(19) United States
(12) Patent Application Publication
(10) Pub. No.: US 2014/0144445 A1
(43) Pub. Date: May 29, 2014

(54) Substantially Constant Positive Airway Pressure Systems and Methods for Treating Sleep Apnea, Snoring, and Other Respiratory Disorders

(71) Applicants: NATHANIEL L BOWDITCH, Menlo Park, CA (US); Thomas G. Goff, Mountain View, CA (US)

(72) Inventors: NATHANIEL L BOWDITCH, Menlo Park, CA (US); Thomas G. Goff, Mountain View, CA (US)

(73) Assignee: Hancock Medical, Menlo Park, CA (US)

(21) Appl. No.: 12/986,808

(22) Filed: Jan. 7, 2011

Related U.S. Application Data
(63) Continuation-in-part of application No. 12/655,829, filed on Jan. 8, 2010, now Pat. No. 8,517,017.
(60) Provisional application No. 61/402,496, filed on Aug. 31, 2010.
(60) Provisional application No. 61/143,371, filed on Jan. 8, 2009.

(51) Int. Cl. A61M 16/20; A61M 16/06; A61M 16/08; A61M 16/00

(52) U.S. Cl. CPC A61M 16/208; A61M 16/0051; A61M 16/0066; A61M 16/06; A61M 16/0875
USPC 128/204.23

(57) ABSTRACT

Substantially constant positive airway pressure systems and methods mediate the variations in pressures that occur within a conventional CPAP mask during inhalation and exhalation cycles, and thereby reduce discomfort.

### RELATED APPLICATION

This application claims the benefit of U.S. Provisional Patent Application Ser. No. 61/402,496, filed Aug. 31, 2010, and entitled “Substantially Constant Positive Airway Pressure Systems and Methods for Treating Sleep Apnea, Snoring, and Other Respiratory Disorders,” which is incorporated herein by reference. This application is also a continuation-in-part of co-pending U.S. patent application Ser. No. 12/655,829, filed Jan. 8, 2010, entitled “Self-Contained, Intermittent Positive Airway Pressure Systems and Methods for Treating Sleep Apnea, Snoring, and Other Respiratory Disorders,” which claims the benefit of U.S. Provisional Patent Application Ser. No. 61/143,371, filed Jan. 8, 2009, and entitled “Devices and Methods for Treating Respiratory Disorders,” which are also incorporated herein by reference.

### FIELD OF THE INVENTION

The invention generally relates to respiration aids to prevent partial or complete airway blockage during sleep, or other respiratory disorders. The invention also generally relates to positive airway pressure systems and methods.

### BACKGROUND OF THE INVENTION

During sleep, all muscles, including those of the upper airway, lose tone and relax. Obstructive Sleep Apnea (OSA) occurs when tissue blocks the upper airway during sleep. This will cause a drop in blood oxygen and a rise in blood carbon dioxide. The brain will sense these changes, and awaken the person enough to restore muscle tone to the structures of the upper airway, and the airway will reopen.

The severity of OSA is determined by the number of blockages per hour of sleep, also called the apnea-hypopnea index (AHI). These include complete blockages (apneas) and partial blockages (hypopneas). The severity of OSA, as determined by a sleep study, is classified as follows:

| Severity | Blockages per Hour (AHI) |
|---|---|
| Mild | 5-15 |
| Moderate | 15-30 |
| Severe | 30+ |

OSA disrupts restorative sleep. Chronic fatigue has long been recognized as the hallmark of OSA. But more recently, large clinical studies have shown a strong link between OSA and stroke and death. This link is independent of other risk factors for cardiovascular disease such as hypertension, obesity, high cholesterol, smoking and diabetes.

### Current Therapies

Several structures can cause blockage of the upper airway: the tongue, the soft palate, the lateral walls of the pharynx, the tonsils and the epiglottis. In most patients, the blockage is caused by a combination of these anatomical structures.

Many procedures and devices have been used to stabilize, modify or remove tissue in the airway to treat OSA. In uvulopalatopharygoplasty (UPPP), the uvula, part of the soft palate and the tonsils are removed. The Repose stitch is used to tie the tongue to the mandible to prevent its posterior movement. Oral appliances move the mandible forward (very slightly) to create more space in the airway.

None of these approaches has achieved much more than a 50% success rate, with success defined as a 50% decrease in AHI to a score below 20. The limited success of these approaches likely stems from the fact that they don't address all anatomical sources of a blockage.

The most widely used therapy for OSA is Continuous Positive Airway Pressure, or CPAP. With CPAP, a bedside console provides a continuous flow of air through a connecting tube to a mask that forms an airtight seal around the nose or nose and mouth. There is an exit hole for the air (both from the compressor and that exhaled by the patient), usually near the junction of the tubing to the compressor and the mask. The compressor within the console spins at a constant level, set by a medical professional, to achieve a sufficient pressure to maintain airway patency during sleep. The pressure provided by CPAP (at a level set by a medical professional) needs to be sufficient to prevent airway collapse during the most vulnerable point in the respiratory cycle, the peak of inhalation.

But even though the compressor spins at a constant speed, the pressure achieved in the mask and in the airway will vary through the respiratory cycle. As a patient with CPAP inhales, pressure in the mask (and the upper airway) will drop. When a patient exhales, pressure will increase. This is because a CPAP patient is drawing from and giving to a limited reservoir of air within the mask and tube. The exit hole and compressor in this system cannot maintain a constant pressure in the mask as a person breathes. Because inhalation lowers pressure in the mask, this will mean the pressure provided by the compressor will be higher than necessary to prevent airway collapse during the rest of the respiratory cycle.

Contrast the CPAP situation to the situation of a person who is not using CPAP. This person draws air from a room and exhales into a room. Because the reservoir of air is so large (i.e., the volume of the room) relative to the volume of the lungs, the subtraction of air from the room (during inhalation) and the addition of air to the room (during expiration) have little effect on the air pressure within the room (and it is largely offset by the expansion and contraction of the torso). Thus a person breathing normally experiences very little variation in air pressure in the proximal portions of the airway (the nose and the mouth).

However, for the reasons explained, a patient with CPAP will not only experience air pressures higher than atmospheric pressure (because of the compressor), the patient will also experience more pressure variation throughout the respiratory cycle than someone who is not using CPAP.

Baroreceptors in the nasal passages are very sensitive to pressure changes. The more the pressure in the nasal passages varies through the respiratory cycle, the less comfortable the patient will be, and the more likely to wake up during sleep, thus defeating the purpose of CPAP.

Roughly half of all patients who try CPAP are unable to sleep with it. One aspect of CPAP that patients dislike arises from the discomfort caused by experiencing variable pressures within the CPAP mask. These make CPAP less comfortable, and contribute to the poor compliance with CPAP.

### Summary of the Technical Features of the Invention

The invention provides substantially constant positive airway pressure systems and methods that mediate the variations in pressures that occur within a conventional CPAP mask during inhalation and exhalation cycles, and thereby reduce discomfort.

In one embodiment, the system and methods include one or more pressure and/or flow sensors communicating with the interior of the CPAP mask. A master controller is coupled to the pressure and/or flow sensors. The master controller operates the CPAP air compressor according to pre-programmed rules (executing prescribed control algorithms) in response to pressure and/or flow conditions sensed by the pressure and/or flow sensors. According to the pre-programmed rules, the master controller operates the air compressor to achieve essentially constant pressure conditions within the mask during inhalation and exhalation, or pressure conditions within the mask that do not exceed or fall below a specified minimal range of pressures.

In another embodiment, the systems and methods include one or more one-way relief valves on the CPAP mask, or tubing connecting the compressor and the mask or on the compressor. The relief valve or valves are closed when pressure in the mask is below a specified magnitude. When pressure in the mask is at or above the specified magnitude, the relief valve or valves open in a one way flow direction to vent air out of the mask, thereby relieving the pressure until it drops below the specified magnitude, at which time the relief valve or valves close. The relief valve or valves accommodate airflow sufficient to allow quick pressure relief within the mask. Alternatively, the relief valves can comprise electrically actuated valves controlled by the master controller.

The technical features of the substantially constant positive airway pressure systems and methods would allow a CPAP device to maintain a narrower pressure range within the mask than current CPAP devices can. This would be more comfortable for the wearer, and should improve compliance.

### BRIEF DESCRIPTION OF THE DRAWINGS

FIG. 1 is a diagrammatic view of a conventional CPAP system.

FIG. 2 is a graph showing the variation of pressure within a mask when coupled to the conventional CPAP system shown in FIG. 1 during an individual's inhalation and exhalation cycles.

FIG. 3 is a diagrammatic view of one embodiment of a substantially constant positive airway pressure system that embodies technical features of the invention.

FIG. 4 is a graph showing the substantially constant pressure maintained within a mask when coupled to the substantially constant positive airway pressure system shown in FIG. 3 during an individual's inhalation and exhalation cycles.

FIG. 5 is a diagrammatic view of another embodiment of a substantially constant positive airway pressure system that embodies technical features of the invention.

FIGS. 6A and 6B show the operation of a one way pressure relief valve that the substantially constant positive airway pressure system shown in FIG. 5 incorporates.

FIG. 7 is a graph showing the substantially constant pressure maintained within a mask when coupled to the substantially constant positive airway pressure system shown in FIG. 5 during an individual's inhalation and exhalation cycles.

### DESCRIPTION OF THE PREFERRED EMBODIMENTS

Although the disclosure hereof is detailed and exact to enable those skilled in the art to practice the invention, the physical embodiments herein disclosed merely exemplify the invention, which may be embodied in other specific structures. While the preferred embodiment has been described, the details may be changed without departing from the invention, which is defined by the claims.

### I. The Problem: An Overview

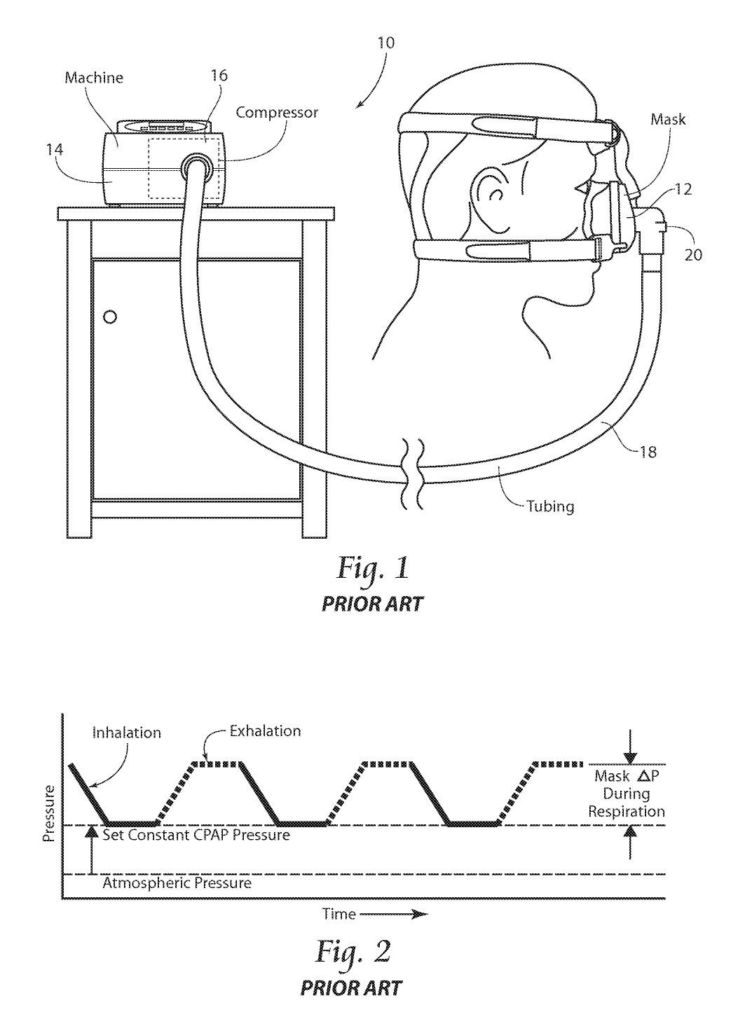

FIG. 1 shows a conventional CPAP system 10. A conventional CPAP system 10 consists of three main components: an airtight mask 12 fitting in or over the nose or nose and mouth; an air pressurizing console 14 that includes an air compressor 16; and a tube 18 connecting the two. There is an exit hole 20 for the air (both from the compressor 16 and that exhaled by the patient), usually near the junction of the tube 18 to the compressor 16 and the mask 12.

The air compressor 16 spins at a constant speed to provide a flow of air at a constant pressure to the mask 12. The magnitude of the speed (and thus the pressure) is set by a medical professional to prevent airway collapse during the most vulnerable point in the respiratory cycle, which is at the peak of inhalation.

Because the air compressor 16 spins at a constant speed to provide a flow of air at a constant pressure to the mask 12, the pressure within the mask 12 and in the airway will vary as a result of the individual's inhalation and exhalation into the mask 12 during the respiratory cycle. This variation is shown in FIG. 2.

As the patient wearing the mask 12 inhales, pressure in the mask 12 (and the upper airway) will drop, because inhalation removes a volume of air from the mask 12 into the airway. The air compressor 16 is set to assure that, during inhalation, the pressure in the mask 12 and the airway nevertheless remains at a high enough level so that airway does not collapse; in effect, the air compressor inflates the airway. Thus, the pressure sensed in the mask 12 by baroreceptors in the individual's nasal passages during inhalation will be higher than would be sensed if the mask was not worn (and the airway perhaps collapsed).

When the patient exhales, pressure in the mask 12 will increase, because exhalation adds the exhaled volume of air to volume of pressurized air already supplied to the mask 12. Because the air compressor 16 is spinning at the same speed during exhalation as it is during inhalation, the pressure in the mask 12 sensed by baroreceptors in the individual's nasal passages during exhalation will also be higher than would be sensed if the mask 12 was not worn. That is because the pressure provided by the compressor 16 must compensate for a decrease in mask pressure during inhalation to prevent airway collapse, so the pressure provided by the compressor 16 to the mask 12 during the rest of the respiratory cycle will of course be higher than necessary to prevent airway collapse.

Thus, during CPAP, not only are the absolute pressures sensed by baroreceptors in the individual's nasal passages higher, particularly during exhalation, the variation in these pressures that occur between inhalation and exhalation are far more noticeable when the mask 12 is worn, than when breathing without a mask 12 in an ambient atmosphere. During CPAP, both the sensed existence of higher absolute pressures in the mask 12 and the heightened variation of these pressures in the mask 12 during the respiration cycle can lead to discomfort.

### II. Feedback Controlled Substantially Constant Mask Pressure

FIG. 3 shows a substantially constant positive airway pressure system 22 for treating sleep apnea, snoring, and other respiratory disorders. The system 22 comprises an airtight mask 24 fitting in or over the nose or nose and mouth; an air pressurizing console 26 that includes an air compressor 28 coupled to a master controller 30 and tubing 32 connecting the two.

According to the invention, the system includes one or more pressure and/or flow sensors 34 that communicate with the interior of the mask. The one or more pressure and/or flow sensors 34 can be carried within the mask 24 itself, or in the tubing 32 that leads to the mask, or a combination thereof.

The master controller 30 is coupled to the pressure and/or flow sensors 34. The master controller 30 operates the air compressor 28 according to pre-programmed rules (executing prescribed control algorithms) in response to pressure and/or flow conditions sensed by the pressure and/or flow sensors 34 communicating with the mask 24. According to the pre-programmed rules, the master controller 30 operates the air compressor 28 to achieve substantially constant pressure conditions within the mask 24 during inhalation and exhalation, or pressure conditions within the mask 24 that do not exceed or fall below a specified minimal range of pressures.

A caregiver can input through a user interface a desired pressure or pressure range to be maintained within the mask 24. The sensors 34 communicating with the mask 24 provide sensed condition signals to the master controller 30. The master controller 30 operates the air compressor 28 according to pre-programmed rules in response, at least in part, to the sensed condition signals in a controlled and coordinated fashion, to maintain the desired substantially constant pressure or pressure range within the mask 24. For example, if the air pressure sensed in the mask 24 falls below that range or level, the master controller 30 commands the compressor 28 to speed up to increase the pressure. If the air pressure sensed in the mask 24 rises above that range or level, the master controller 30 commands the compressor 28 to slow down, allowing pressure to drop.

The preprogrammed rules can provide control commands that are proportional to sensed absolute deviations from control threshold(s). Alternatively, the preprogrammed rules can provide integral or derivative control commands that are based upon the changes in the deviations over time (increasing? or decreasing?) as well as the rate of the changes in the deviations (i.e., by sensing whether the deviations are getting larger or smaller over time and by how much).

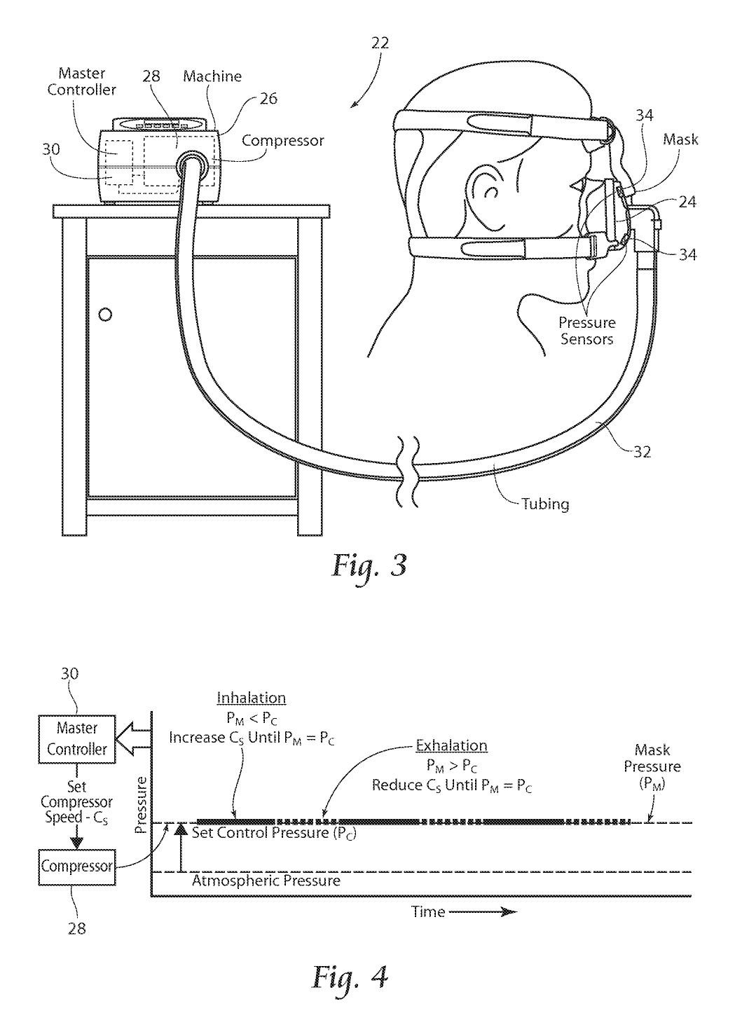

FIG. 4 demonstrates how the master controller 30 maintains the substantially constant pressure or pressure range within the mask 24 in response to pressure and/or flow conditions sensed by the pressure and/or flow sensors 34 communicating with the mask 24.

The system 22 that embodies the technical features just described can be incorporated into either a traditional CPAP system, or one in which the entire system (including the compressor and power source) is worn by the patient. A caregiver can input to the master controller 30 a desired pressure or pressure range for the mask 24, and the master controller 30 maintains the pressure or pressure range in the mask 24 in the manners just described.

Such a system 22 would also work if the expiration opening(s) in the CPAP mask 24 were small enough to significantly restrict the flow of expired air, thereby using the patient's own expiratory pressure to increase pressure in the mask 24 and upper airway during the expiratory phase of the breathing cycle. The pressure and/or flow sensor(s) 34 communicating with the mask 24 would maintain pressure in the mask 24 at a constant level or within a specified range, while a combination of the compressor 28 and the force of expiration both contribute to the pressure in the mask 24.

The preprogrammed rule of the master controller 30 could also adapt to the wearer's breathing pattern. For example, if pressure routinely dropped near the end of each exhalation, the master controller 30 could direct the air compressor 28 to speed up in anticipation of this drop, thereby preventing the pressure in the mask 24 from dropping below the specified value or range.

### III. One-Way Relief Valves to Provide Substantially Constant Mask Pressure

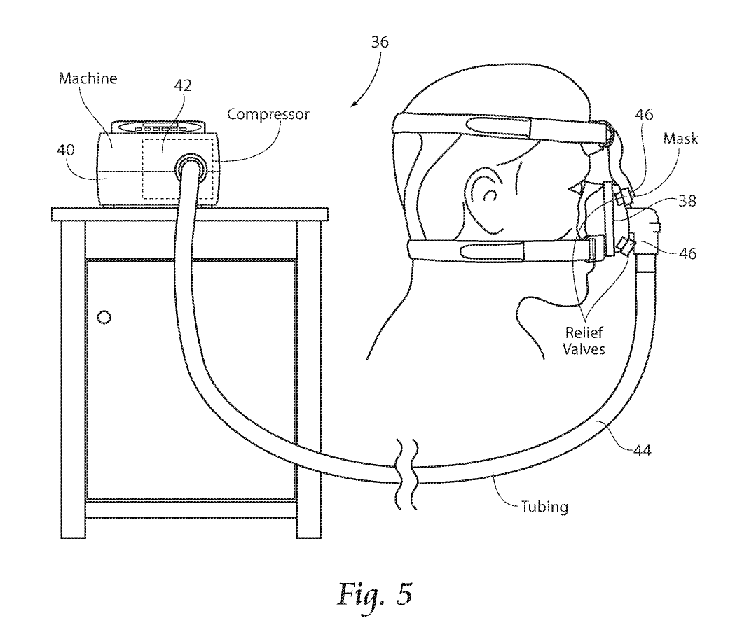

FIG. 5 shows another embodiment of a constant positive airway pressure system 36 for treating sleep apnea, snoring, and other respiratory disorders. The system 36 comprises an airtight mask 38 fitting in or over the nose or nose and mouth; an air pressurizing console 40 that includes an air compressor 42; and tubing 44 connecting the two.

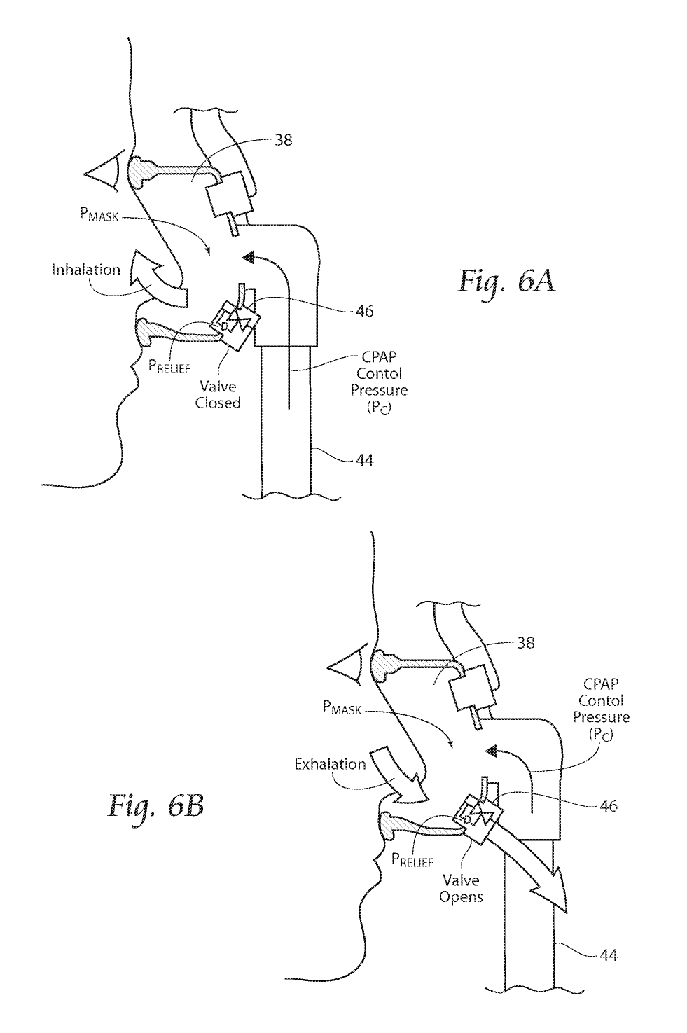

According to the invention (see FIGS. 6A/B and 7), the system 36 includes one or more one-way relief valves 46 on the CPAP mask 38, or tubing connecting the compressor 42 and the mask 38, or on the compressor 42. The relief valve or valves 46 are normally closed when pressure in the mask 38 is below a specified magnitude (as FIG. 6A shows). When pressure in the mask 38 is at or above the specified magnitude, the relief valve or valves 46 open in a one way flow direction to vent air (see FIG. 6B), thereby relieving the pressure until it drops below the specified magnitude, at which time the relief valve or valves 46 close. The relief valve or valves 46 accommodate airflow sufficient to allow quick pressure relief within the mask 38.

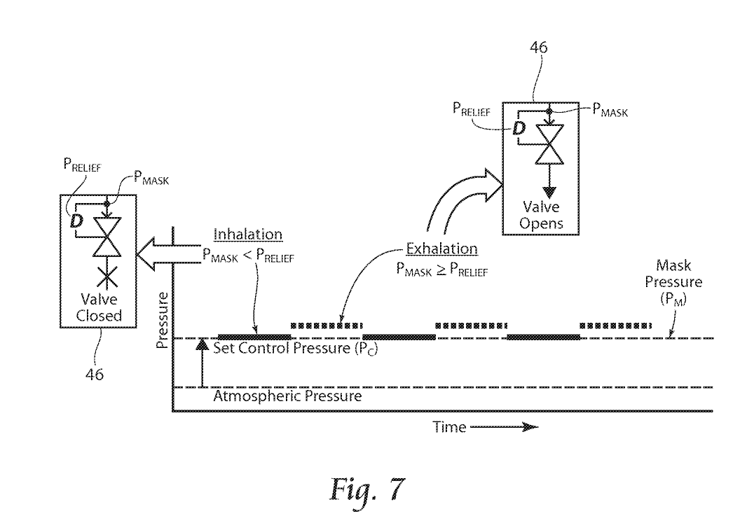

As FIG. 7 shows, the opening and closing of the relief valve or valves 46 maintain a narrow pressure range within the CPAP mask 38 through the respiratory cycle without having to alter the speed of the compressor 42. As a result, the variations in pressure within the mask 38 would be minimal (i.e. less disruptive to the individual during sleep) and the pressure at all times could be the minimum needed to maintain airway patency.

The specified magnitude of pressure at which a given relief valve 46 opens could be set by a caregiver, e.g., using a special tool. Alternatively, a variety of masks could be made, each with a relief valve or valves 46 preset to a specified pressure (e.g., 8 cm H2O, 9 cm H2O, 10 cm H2O, etc.). Having different masks would be less expensive to manufacture than having an adjustable relief valve, and much less expensive than having a pressure or flow sensor in the mask 38 which would determine the compressor speed.

The compressor 42 could be set at a constant speed to provide the critical pressure needed to maintain airway patency during peak inhalation. At this point the relief valve or valves 46 would be closed or open only minimally. When the patient is exhaling, the increased pressure in the mask 38 would open the relief valve, and pressure within the mask 38 would not increase much. The valve would also be open somewhat when the patient is neither inhaling nor exhaling.

The relief valve or valves 46 could be either an “all or nothing” valve (either fully open or fully closed), or it could be a variable relief valve that opens more as pressure increases.

Alternatively, or in combination, the relief valve or valves 46 can comprise a low-power electrically or pneumatically actuated valve, which are coupled to the master controller 30 and which open and close according to preprogrammed rules in the master controller 30. As governed by the preprogrammed rules, the “smart” relief valves 46 open and close at different times during the respiratory cycle, so that air from the compressor 42 is not vented to the atmosphere unintentionally.

The system can further include a passive valve that opens to let air into the mask 38 if the pressure in the mask 38 falls below room pressure. This valve would add a measure of safety should the compressor 42 fail or malfunction.

Such a system could be employed in either a traditional CPAP system, or one in which the entire system (including the compressor 42 and power source) is worn by the patient. This embodiment of the invention would allow a CPAP device to maintain a narrower pressure range within the mask 38 than current CPAP devices can. This would be more comfortable for the wearer, and thus improve compliance.

The technical features just described would allow a CPAP device to maintain a narrower pressure range within the mask 38 than current CPAP devices can. This would be more comfortable for the wearer, and should improve compliance. The above-described embodiments of this invention are merely descriptive of its principles and are not to be limited. The scope of this invention instead shall be determined from the scope of the following claims, including their equivalents.

We claim:

1. A system comprising
an air compressor operable to supply air pressure at a positive pressure magnitude,
a delivery device sized and configured to be worn by an individual in airtight communication with an individual's airway and being coupled to the air compressor to deliver the air pressure from the air compressor into the airway while sleeping,
a sensing component communicating with the delivery device and being sized and configured to sense pressure and/or flow conditions within the delivery device, and
a controller coupled to the air compressor and the sensing component including pre-programmed rules to operate the air compressor in response to the sensed pressure and/or flow conditions to maintain a prescribed substantially constant positive pressure magnitude or a prescribed range of positive pressure magnitudes within the delivery device during inhalation and exhalation phases of the individual's respiratory cycle.

2. A system according to claim 1
wherein the sensing component is carried within the delivery device.

3. A system according to claim 1
wherein the sensing component is carried within tubing that couples the delivery device to the air compressor.

4. A system according to claim 1
wherein the delivery device comprises a mask.

5. A system according to claim 1
wherein the air compressor is operative to spin at a rotational speed that provides a flow of air at the positive pressure magnitude, and
wherein the pre-programmed rules vary the rotational speed of the air compressor in response to the sensed pressure conditions.

6. A system according to claim 5
wherein when the sensed pressure and/or flow conditions within the delivery device fall below a threshold condition, the pre-programmed rules increases the rotational speed of the air compressor to increase the positive pressure magnitude.

7. A system according to claim 5
wherein when the sensed pressure and/or flow conditions within the delivery device rises above a threshold condition, the pre-programmed rules decreases the rotational speed of the air compressor to decrease the positive pressure magnitude.

8. A system according to claim 1
wherein pre-programmed rules generate control commands in response to the sensed pressure and/or flow conditions that are proportional to deviations from a prescribed control threshold.

9. A system according to claim 1
wherein pre-programmed rules generate integral and/or derivative control commands in response to the sensed pressure and/or flow conditions that are based upon changes in deviations from a prescribed control threshold and/or rate of change in deviations from a prescribed control threshold.

10. A system according to claim 1
wherein the air compressor and delivery device comprise a CPAP system.

11. A system according to claim 1
wherein the air compressor, delivery device, and controller comprise an integrated unit worn by the individual.

12. A system according to claim 1
wherein the delivery device includes expiration openings sized and configured to restrict the flow of expired air during the exhalation phase of the individual's respiratory cycle, thereby increasing the positive pressure magnitude within the delivery device.

13. A system according to claim 1
wherein the pre-programmed rules adapt to the individual breathing pattern.

14. A method comprising
providing air compressor operable to supply air pressure at a positive pressure magnitude,
providing a delivery device sized and configured to be worn by an individual in airtight communication with an individual's airway,
coupling the delivery device to the air compressor to deliver the air pressure from the air compressor into the airway while sleeping,
sensing pressure and/or flow conditions within the delivery device, and
operating the air compressor in response to the sensed pressure and/or flow conditions to maintain a prescribed substantially constant positive pressure magnitude or a prescribed range of positive pressure magnitudes within the delivery device during inhalation and exhalation phases of the individual's respiratory cycle.

15. A method according to claim 14
wherein, when the sensed pressure and/or flow conditions within the delivery device fall below a threshold condition, the air compressor is operated to increase the positive pressure magnitude.

16. A method according to claim 14
wherein, when the sensed pressure and/or flow conditions within the delivery device rises above a threshold condition, the air compressor is operated to decrease the positive pressure magnitude.

17. A method according to claim 14
wherein operating the air compressor includes generating control commands in response to the sensed pressure and/or flow conditions that are proportional to deviations from a prescribed control threshold.

18. A method according to claim 14
wherein operating the air compressor includes generating integral and/or derivative control commands in response to the sensed pressure and/or flow conditions that are based upon changes in deviations from a prescribed control threshold and/or rate of change in deviations from a prescribed control threshold.

19. A method according to claim 14
wherein operating the air compressor includes adapting to the individual breathing pattern.

20. A method according to claim 14
further including restricting the flow of expired air within the delivery device during the exhalation phase of the individual's respiratory cycle, thereby increasing the positive pressure magnitude within the delivery device.

21. A system comprising
an air compressor operable to supply air pressure at a positive pressure magnitude,
a delivery device sized and configured to be worn by an individual in airtight communication with an individual's airway and being coupled to the air compressor to deliver the air pressure from the air compressor into the airway while sleeping,
a one-way relief valve component communicating with the delivery device and being sized and configured to open communication with ambient air when pressure conditions within the delivery device equal or exceed a specified threshold and close communication with ambient air when pressure conditions within the delivery device fall below the specified threshold, thereby maintaining a prescribed substantially constant positive condition within the delivery device during inhalation and exhalation phases of the individual's respiratory cycle.

22. A system according to claim 21
wherein the one-way relief valve component is carried within the delivery device.

23. A system according to claim 21
wherein the one-way relief valve component is carried within tubing that couples the delivery device to the air compressor.

24. A system according to claim 21
wherein the delivery device comprises a mask.

25. A system according to claim 21
wherein the air compressor is operative to spin at a substantially constant rotational speed that provides a substantially constant positive pressure magnitude.

26. A system according to claim 25
wherein the substantially constant rotational speed provides a substantially constant positive pressure magnitude that maintains airway patency during peak inhalation.

27. A system according to claim 21
wherein the one-way relief valve component comprises a normally closed one-way valve.

28. A system according to claim 21
wherein the one-way relief valve component comprises a fully opened condition and a fully closed condition.

29. A system according to claim 28
wherein the one-way relief valve component comprises a range of partially open conditions between the fully opened condition and the fully closed condition that increase according to increasing magnitude of positive pressure magnitudes within the delivery device.

30. A system according to claim 21
wherein the one-way relief valve component comprises an electrically or pneumatically actuated valve.

31. A system according to claim 30
further including a controller coupled to the electrically or pneumatically actuated valve that opens and closes the electrically or pneumatically actuated valve according to pre-programmed rules.

32. A system according to claim 31
wherein the pre-programmed rules open and close the electrically or pneumatically actuated valve at different times during the individual's respiratory cycle to minimize opening of the electrically or pneumatically actuated valve.

33. A system according to claim 21
further including a passive valve component communicating with the delivery device and being sized and configured to open communication with ambient atmosphere when the pressure condition within the delivery device falls below ambient air pressure.

34. A system according to claim 21
wherein the air compressor and delivery device comprise a CPAP system.

35. A system according to claim 21
wherein the air compressor, delivery device, and one-way relief valve component comprise an integrated unit worn by the individual.

36. A system according to claim 21
wherein the one-way relief valve component comprises an electrically or pneumatically actuated valve,
further including a controller coupled to the electrically or pneumatically actuated valve that opens and closes the electrically or pneumatically actuated valve according to pre-programmed rules, and
wherein the air compressor, delivery device, one-way relief valve component, and controller comprise an integrated unit worn by the individual.

37. A method comprising
providing a delivery device sized and configured to be worn by an individual in airtight communication with an individual's airway and being coupled to an air compressor to deliver positive air pressure into the airway while sleeping, and
maintaining a prescribed substantially constant positive condition within the delivery device during inhalation and exhalation phases of the individual's respiratory cycle by (i) operating a one-way relief valve component communicating with the delivery device to open communication with ambient air when pressure conditions within the delivery device equal or exceed a specified threshold, and (ii) operating the one-way relief valve component to close communication with ambient air when pressure conditions within the delivery device fall below the specified threshold.

38. A method according to claim 37
further including operating the air compressor to supply positive pressure at a substantially constant positive pressure magnitude into the delivery device.

39. A method according to claim 38
wherein the substantially constant positive pressure magnitude is selected that maintains airway patency during peak inhalation.

40. A method according to claim 37
operating a passive valve component communicating with the delivery device to open communication with ambient atmosphere when the pressure condition within the delivery device falls below ambient air pressure.
